(19) United States
(12) Patent Application Publication
(10) Pub. No.: US 2010/0160644 A1
(43) Pub. Date: Jun. 24, 2010

(54) NOVEL PROCESSES OF MAKING HYDROXY-1-AZO-DERIVATIVES AS TPO MIMETICS

(76) Inventor: Jerome Francis Hayes, Kent (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/597,181

(22) PCT Filed: Apr. 23, 2008
(86) PCT No.: PCT/US08/61225

§ 371 Date: Feb. 19, 2010

Related U.S. Application Data
(60) Provisional application No. 60/913,601, filed on Apr. 24, 2007.

(51) Int. Cl. C07D 403/12

(52) U.S. Cl. 548/253

(57) ABSTRACT

Invented are novel processes of making hydroxy-1-azo-benzene derivatives as TPO mimetics. Also invented are novel intermediates used in the novel processes. Also invented are pharmaceutical compositions comprising compounds made by novel processes.

### FIELD OF THE INVENTION

This invention relates to processes of making thrombopoietin (TPO) mimetics which are useful as promoters of thrombopoiesis and megakaryocytopoiesis.

### BACKGROUND OF THE INVENTION

Megakaryocytes are bone marrow-derived cells, which are responsible for producing circulating blood platelets. Although comprising <0.25% of the bone marrow cells in most species, they have >10 times the volume of typical marrow cells. See Kuter et al. Proc. Natl. Acad. Aci. USA 91: 11104-11108 (1994). Megakaryocytes undergo a process known as endomitosis whereby they replicate their nuclei but fail to undergo cell division and thereby give rise to polypoid cells. In response to a decreased platelet count, the endomitotic rate increases, higher ploidy megakaryocytes are formed, and the number of megakaryocytes may increase up to 3-fold. See Harker J. Clin. Invest. 47: 458-465 (1968). In contrast, in response to an elevated platelet count, the endomitotic rate decreases, lower ploidy megakaryocytes are formed, and the number of megakaryocytes may decrease by 50%.

The exact physiological feedback mechanism by which the mass of circulating platelets regulates the endomitrotic rate and number of bone marrow megakaryocytes is not known. The circulating thrombopoietic factor involved in mediating this feedback loop is now thought to be thrombopoietin (TPO). More specifically, TPO has been shown to be the main humoral regulator in situations involving thrombocytopenia. See, e.g., Metcalf Nature 369:519-520 (1994). TPO has been shown in several studies to increase platelet counts, increase platelet size, and increase isotope incorporation into platelets of recipient animals. Specifically, TPO is thought to affect megakaryocytopoiesis in several ways: (1) it produces increases in megakaryocyte size and number; (2) it produces an increase in DNA content, in the form of polyploidy, in megakaryocytes; (3) it increases megakaryocyte endomitosis; (4) it produces increased maturation of megakaryocytes; and (5) it produces an increase in the percentage of precursor cells, in the form of small acetylcholinesterase-positive cells, in the bone marrow.

Because platelets (thrombocytes) are necessary for blood clotting and when their numbers are very low a patient is at risk of death from catastrophic hemorrhage, TPO has potential useful application in both the diagnosis and the treatment of various hematological disorders, for example, diseases primarily due to platelet defects (see Harker et al. Blood 91: 4427-4433 (1998)). Ongoing clinical trials with TPO have indicated that TPO can be administered safely to patients (see Basser et al. Blood 89: 3118-3128 (1997); Fanucchi et al. New Engl. J. Med. 336: 404-409 (1997)). In addition, recent studies have provided a basis for the projection of efficacy of TPO therapy in the treatment of thrombocytopenia, and particularly thrombocytopenia resulting from chemotherapy, radiation therapy, or bone marrow transplantation as treatment for cancer or lymphoma. (See Harker, Curr. Opin. Hematol. 6: 127-134 (1999)).

The gene encoding TPO has been cloned and characterized. See Kuter et al., Proc. Natl. Acad. Sci. USA 91: 11104-11108 (1994); Barley et al., Cell 77: 1117-1124 (1994); Kaushansky et al., Nature 369:568-571 (1994); Wendling et al., Nature 369: 571-574 (1994); and Sauvage et al., Nature 369: 533-538 (1994). Thrombopoietin is a glycoprotein with at least two forms, with apparent molecular masses of 25 kDa and 31 kDa, with a common N-terminal amino acid; sequence. See, Baatout, Haemostasis 27: 1-8 (1997); Kaushansky, New Engl. J. Med. 339: 746-754 (1998). Thrombopoietin appears to have two distinct regions separated by a potential Arg-Arg cleavage site. The amino-terminal region is highly conserved in man and mouse, and has some homology with erythropoietin and interferon-a and interferon-b. The carboxy-terminal region shows wide species divergence.

The DNA sequences and encoded peptide sequences for human TPO receptor (TPO-R; also known as c-mpl) have been described. (See, Vigon et al. Proc. Natl. Acad. Sci. USA 89: 5640-5644 (1992)). TPO-R is a member of the haematopoietin growth factor receptor family, a family characterized by a common structural design of the extracellular domain, including for conserved C residues in the N-terminal portion and a WSXWS motif close to the transmembrane region. (See Bazan Proc. Natl. Acad. Sci. USA 87: 6934-6938 (1990)). Evidence that this receptor plays a functional role in hematopoiesis includes observations that its expression if restricted to spleen, bone marrow, or fetal liver in mice (see Souyri et al. Cell 63: 1137-1147 (1990)) and to megakaryocytes, platelets, and CD34⁺ cells in humans (see Methia et al. Blood 82: 1395-1401 (1993)). Further evidence for TPO-R as a key regulator of megakaryopoiesis is the fact that exposure of CD34⁺ cells to synthetic oligonucleotides antisense to TPO-R RNA significantly inhibits the appearance of megakaryocyte colonies without affecting erythroid or myeloid colony formation. Some workers postulate that the receptor functions as a homodimer, similar to the situation with the receptors for G-CSF and erythropoietin. (see Alexander et al. EMBO J. 14: 5569-5578 (1995)).

The slow recovery of platelet levels in patients suffering from thrombocytopenia is a serious problem, and has lent urgency to the search for a blood growth factor agonist able to accelerate platelet regeneration (see Kuter, Seminars in Hematology, 37: Supp 4: 41-49 (2000)).

It would be desirable to provide compounds which allow for the treatment of thrombocytopenia by acting as a TPO mimetic.

WO01/89457A2 describes certain hydroxy-1-azo-benzene derivatives as TPO mimetics and processes of preparing these compounds. Disclosed in the present invention are novel processes of preparing hydroxy-1-azo-benzene derivatives. Current invention shows advantages over the method described in WO01/89457A2 since several problematic transformations are avoided. The current invention avoids demethylation of anisole which gives bromomethane as a highly toxic and volatile side product, the potentially hazardous nitration and the difficult hydrogenolysis of the aryl chloride. The starting materials 2-nitrophenol and 3-cyanoboronic acid are readily available.

### DETAILED DESCRIPTION OF THE INVENTION

This invention relates to novel processes of making hydroxy-1-azo-benzene derivatives which have utility as TPO mimetics. This invention also relates to novel intermediates used in the novel processes.

This invention relates to a process for the preparation of a compound of formula I:

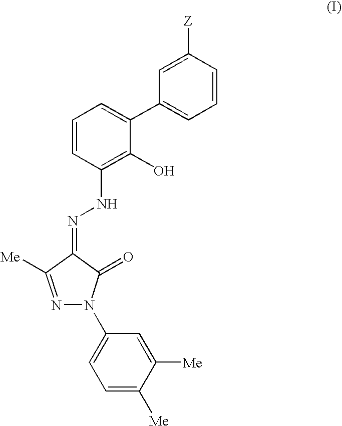

wherein Z is —COOH or tetrazol-yl;
or a pharmaceutically acceptable salt thereof, which
comprises the steps of:
(1) treating a compound of formula II:

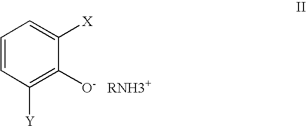

wherein X is Cl, Br, I, or a group suitable for coupling with Boronic acids, Y is NO₂ or NH₂, and R is alkyl or substituted alkyl;
with a Boronic acid in a solvent to form a compound of formula III:

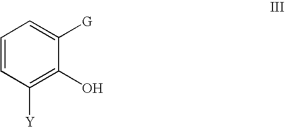

wherein Y is NH₂ or NO₂, and G is substituted aryl.
(2) converting a compound of formula III to a compound of formula I; and thereafter optionally forming a pharmaceutically acceptable salt thereof.

This invention also relates to a process for the preparation of a compound of formula I:

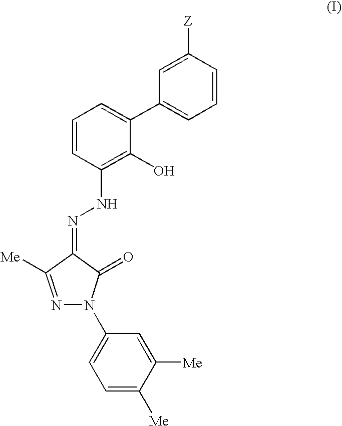

wherein Z is tetrazol-yl;
or a pharmaceutically acceptable salt thereof, which
comprises the steps of:
(1) treating a compound of formula. II:

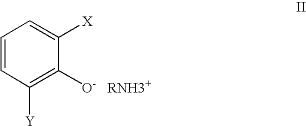

wherein X is Br, Y is NO₂, and R is alkyl or substituted alkyl;
with a Boronic acid in a solvent to form a compound of formula III:

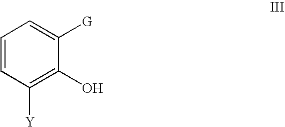

wherein Y is NO₂, and G is substituted aryl.
(2) converting a compound of formula III to a compound of formula I; and thereafter optionally forming a pharmaceutically acceptable salt thereof.

This invention also relates to a process for the preparation of a compound of formula I:

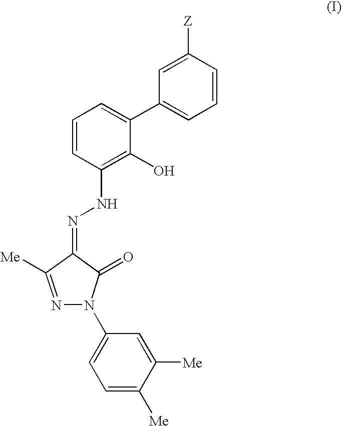

wherein Z is tetrazol-yl;
or a pharmaceutically acceptable salt thereof, hydrate, solvate or produg thereof, which
comprises the steps of:
(1) treating a compound of formula II:

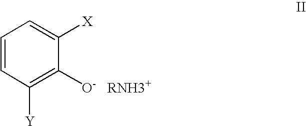

wherein X is Br, Y is NO₂, and R is t-Butyl;
with a Boronic acid in a solvent to form a compound of formula III:

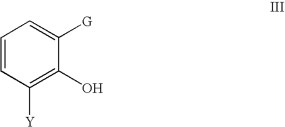

wherein Y is NO₂, and G is substituted aryl.
(2) converting a compound of formula III to a compound of formula I; and thereafter optionally forming a pharmaceutically acceptable salt thereof.

This invention also relates to a process of the preparation of a compound of formula I:

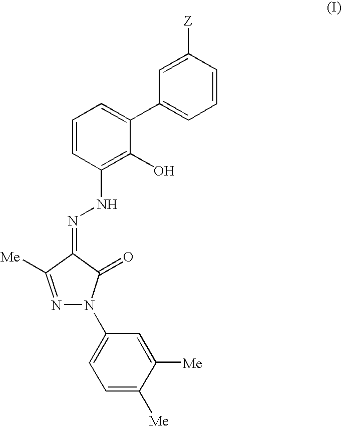

wherein Z is —COOH;
or a pharmaceutically acceptable salt thereof, which
comprises the steps of:
(1) treating a compound of formula II:

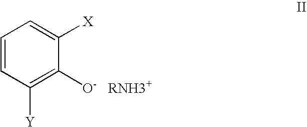

wherein X is Br, Y is NO₂, and R is alkyl or substituted alkyl;
with a Boronic acid in a solvent to form a compound of formula III:

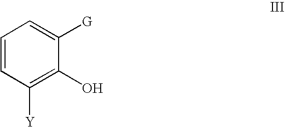

wherein Y is NO₂, and G is substituted aryl.
(2) converting a compound of formula III to a compound of formula I; and thereafter optionally forming a pharmaceutically acceptable salt thereof.

This invention also relates to a process for the preparation of a compound of formula I:

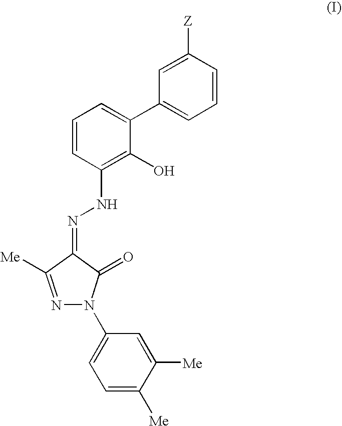

wherein Z is —COOH;
or a pharmaceutically acceptable salt thereof, hydrate, solvate or produg thereof, which
comprises the steps of:
(1) treating a compound of formula II:

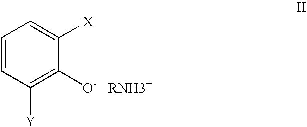

wherein X is Br, Y is NO₂, and R is t-Butyl;
with a Boronic acid in a solvent to form a compound of formula III:

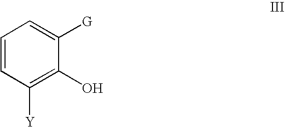

wherein Y is NO₂, and G is substituted aryl.
(2) converting a compound of formula III to a compound of formula I; and thereafter optionally forming a pharmaceutically acceptable salt thereof.

This invention also relates to a compound of formula I prepared by a novel process on a large scale. Compounds prepared on large industrial scales often require a substantially pure starting materials and result in a unique impurity profile.

As used herein, the term “a group suitable for coupling with Boronic acids” is meant a functional group, when attached to a molecule, enables the molecule to undergo a catalytic reaction with a boronic acid and form a bond.

As used herein, the term “large scale” is meant a scale of a series of reactions which produce greater than 50 grams of product, preferably greater than 10 kilogram of product. As used herein, the term “solvent” is meant an organic solvent, inorganic solvent, or a mixture of organic and inorganic solvents at a suitable ratio.

As used herein, the term “substituted” is meant By the term “substituted” as used herein, unless otherwise defined, is meant that the subject chemical moiety has one or more substituents, suitably from one to five substituents, suitably from one to three, selected from the group consisting of: hydrogen, halogen, C1-C6alkyl, amino, trifluoromethyl, —(CH₂)COOH, C3-C7cycloalkyl, aminoalkyl, aryl, heteroaryl, arylalkyl, arylcycloalkyl, heteroarylalkyl, heterocycloalkyl, cyano, hydroxyl, alkoxy, aryloxy, acyloxy, acylamino, arylamino, nitro, oxo, —CO₂R₅₀, and CONR₅₅R₆₀, wherein R50, R55 and R60 are each independently selected from hydrogen, and alkyl; n is 0 to 6.

As used herein, the term “Boronic acid” is meant a compound of the following structure:

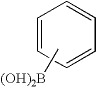

wherein the aromatic ring is optionally substituted.

When Z is —COOH, the salt is suitably Bis-monoethanolamine. When Z is tetrazol-yl, the salt is suitably choline.

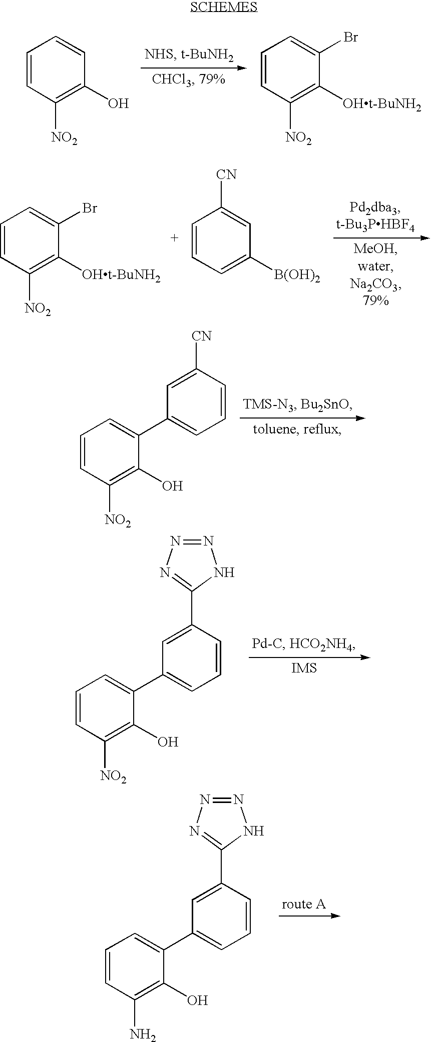

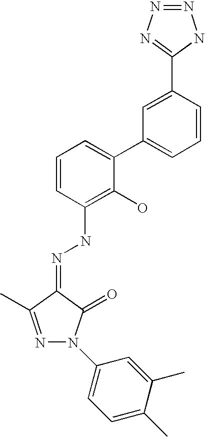

### EXAMPLES

### 2-Bromo-6-Nitrophenol t-butylamine salt

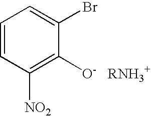

To a suspension of NBS (2.56 g, 14.4 mmol) in chloroform (70 ml) was added t-butylamine (1.50 ml, 14.4 mmol). The resultant solution was cooled to 0-5° C. and 2-nitrophenol (4.00 g, 28.8 mmol) in chloroform (35 ml) was then added over 10 min. The resultant slurry was stirred for 1 h and the product was then filtered off and washed with chloroform (20 ml). The product was obtained as a light orange solid, yield=3.03 g, 73%; δ_{H}(400 MHz, DMSO) 1.25 (9H, s), 5.85 (1H, t), 7.45 (1H, d), 7.65 (1H, d) and 7.9 (3H, s br).

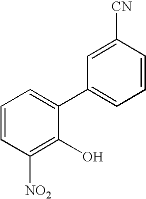

2′-hydroxy-3′-nitro-3-biphenylcarbonitrile To a mixture of 2-bromo-6-nitrophenol t-butylamine salt (1.50 g, 5.20 mmol) and sodium carbonate (0.66 g, 6.2 mmol) in methanol (12 ml) and water (0.48 ml) was added tris dibenzylidine acetone dipalladium (0) (94 mg, 0.103 mmol) and tri t-butylphosphonium tetrafluoroborate (52 mg, 0.179 mmol) under argon. The mixture was heated to reflux (60° C.) for 30 min. after which time 3-cyanoboronic acid (0.83 g, 5.68 mmol) in methanol (12 ml) and water (0.48 ml) was added over 1 h under argon. The reaction was maintained at reflux for 4 h. After this time acetic acid (15 ml) was added and solvent (17 ml) was distilled off. Water (15 ml) was then added over 5 min at 50-70° C. The resultant slurry was allowed to cool to rt and the product was filtered off and washed with water (20 ml) and was sucked dry. The product was obtained as a brown solid (0.98 g, 79%); δ_{H (}400 MHz, DMSO) 7.18 (1H, t), 7.58 (1H, t), 7.75 (1H, dd), 7.90 (2H, m), 8.01 (1H, s), 8.07 (1H, dd) and 10.7 (1H, s br).

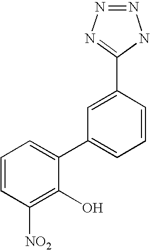

3-nitro-3′-(1H-tetrazol-5-yl)-2-biphenylol To a suspension of SB-817250 (1.00 g, 4.17 mmol) in toluene (10 ml) was added dibutyltinoxide (104 mg, 0.42 mmol) followed by trimethylsilylazide (1.20 ml, 9.14 mmol). The reaction mixture was heated under reflux for 20 h. After this time toluene (10 ml) and methanol (1670 were added at 50-60° C. The reaction mixture was then cooled to room temperature. The dark fine suspension was stirred for 1 h and was then filtered. The product was washed with toluene (4 ml) and was then sucked dry and was obtained as a dark brown solid (0.75 g, 64%); 64400 MHz, DMSO) 7.05 (1H, s), 7.60-7.72 (3H, m), 8.02 (2H, d) and 8.17 (1H, s).

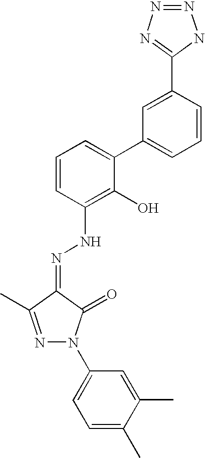

(47)-1-(3,4-dimethylphenyl)-3-methyl-1H-pyrazole-4,5-dione 4-{[2-hydroxy-3′-(1H-tetrazol-5-yl)-3-biphenylyl]hydrazone}
3-nitro-3′-(1H-tetrazol-5-yl)-2-biphenylol was then converted to (4Z)-1-(3,4-dimethylphenyl)-3-methyl-1H-pyrazole-4,5-dione 4-{[2-hydroxy-3′-(1H-tetrazol-5-yl)-3-biphenylyl]hydrazone} using route A described in WO01/89457A2. WO01/89457A2 is incorporated herein by reference.

While the preferred embodiments of the invention are illustrated by the above, it is to be understood that the invention is not limited to the precise instructions herein disclosed and that the right to all modifications coming within the scope of the following claims is reserved.

What is claimed is:

1. A process for the preparation of a compound of formula I: 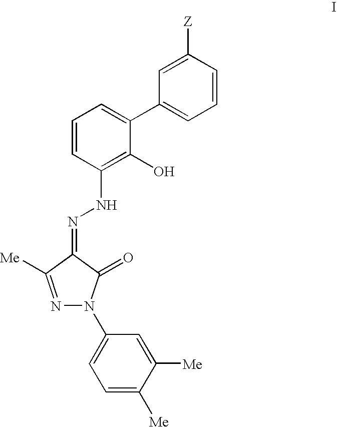
wherein Z is COOH or tetrazol;
or a pharmaceutically acceptable salt thereof, which
comprises the steps of:
(1) treating a compound of formula II: 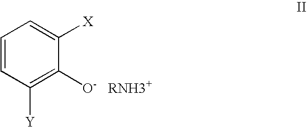
wherein X is Cl, Br, or I, Y is NO₂ or NH₂, and R is alkyl or substituted alkyl;
with a Boronic acid in a solvent to form a compound of formula III: 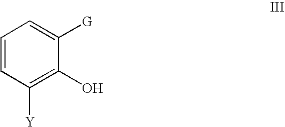
wherein Y is NH₂ or NO₂, and G is substituted aryl.
(2) converting a compound of formula III to a compound of formula I; and
thereafter optionally forming a pharmaceutically acceptable salt thereof.

2. A compound of formula I when prepared on a large scale according to the process of claim 1.

3. A process according to claim 1 wherein X is Br, Y is NO₂, R is t-Butyl, and Z is tetrazol.

4. A process according to claim 1 wherein X is Br, Y is NO₂, R is t-Butyl, and Z is COOH.

5. A compound of formula III when prepared on a large scale according to the process of claim 1, wherein Y is NO₂ or NH₂, and G is: [chemical structure]
